(19) United States
(12) Patent Application Publication
(10) Pub. No.: US 2009/0041816 A1
(43) Pub. Date: Feb. 12, 2009

(54) Coated particles

(75) Inventors: Ivan Petrovich Dotsenko, Moscow (RU); Ann-Marie Williamson, Sharnbrook (GB); Gleb Yakubov, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/220,981

(22) Filed: Jul. 30, 2008

(30) Foreign Application Priority Data
Aug. 1, 2007 (EP) EP07113633

(51) Int. Cl. A61K 9/14; A23P 1/08; A61K 8/00

(52) U.S. Cl. 424/401; 426/98; 426/97; 426/92; 426/303; 424/493; 424/498; 424/494

(57) ABSTRACT

A coated particle is disclosed comprising a core, a first innermost layer coating the core, a second middle layer coating the first innermost layer and a third outermost layer coating the second middle layer, wherein the core comprises at least a water-in-oil emulsion or a fat and/or oil, wherein the first innermost layer comprises at least one emulsifier and, wherein the second middle layer comprises either one or more polyanions or one or more polycations and the third outermost layer comprises one or more polyelectrolytes only of opposing charge to that of the polyanions or polycations of the second middle layer. The inventors have observed that the particle has improved stability, particularly when the particles are small as the forces between said small particles to aggregate is greater and simultaneously provides a delivery vehicle for included actives and/or flavours. A method for the manufacture of the particle is also disclosed as is a product selected from the group consisting of a food product, a home care product, a personal care product and a pharmaceutical product, wherein each product comprises a plurality of the coated particles.

The present invention relates to a coated particle comprising a core, a first innermost layer coating the core, a second middle layer coating the first innermost layer and a third outermost layer coating the second middle layer, wherein the core comprises at least a water-in-oil emulsion or a fat and/or oil, wherein the first innermost layer comprises at least one emulsifier and, wherein the second middle layer consists of either one or more polyanions or one or more polycations and the third outermost layer consists only of one or more polyelectrolytes of opposing charge to that of the polyanions or polycations of the second middle layer. The invention further relates to a process for the manufacture of the said particle.

Coated particles comprising more than one layer are known from the prior art such as in WO 2007/038616 A1. However the number of layers disclosed in the prior art is limited. Furthermore known methods for the manufacture of coated particles do not effect the reversal of charge on the surface of the coated particle required for the adsorption of more than two polyelectrolyte layers.

The goal of the present invention was to provide multilayer coated particles that have a range of benefits including improved emulsion stability and improved deposition and delivery benefits for included actives and flavours.

### BRIEF DESCRIPTION OF THE INVENTION

In a first aspect, the invention provides a coated particle comprising a core, a first innermost layer coating the core, a second middle layer coating the first innermost layer and a third outermost layer coating the second middle layer, wherein the core comprises at least a water-in-oil emulsion or a fat and/or oil, wherein the first innermost layer comprises at least one emulsifier and, wherein the second middle layer comprises either one or more polyanions or one or more polycations and the third outermost layer comprises one or more polyelectrolytes only of opposing charge to that of the polyanions or polycations of the second middle layer.

The inventors have observed that the particle:
a) Has improved stability, particularly when the particles are small as the forces between said small particles to coalesce is greater.
b) Provides a delivery vehicle for included actives and/or flavours.

By the term active or flavour is meant any natural and/or synthetic biologically active ingredients and sensory marker ingredient that can be delivered by the particle of the invention. Example ingredients include saturated and unsaturated fatty acids, essential fatty acids, glycerols, glycerides and their respective derivatives, phospholipids and their respective derivatives, glycolipids, phytosterol, sterol esters such as cholesterol esters and their respective derivatives thereof, phytosterol esters and their respective derivatives thereof, flavour oils such as peppermint, citrus, coconut, or vanilla, antioxidants, vitamins and their derivatives such as retinoids and carotenoids, terpenes and their respective derivatives, alkaloids such as caffeine, flavanoids such as flavan-3-ols, anthocyanins, phenolic acids, polyphenols, amino acids, anti-bacterial and/or anti-fungal agents, colorants, and/or flavourants including trigeminal stimulants. Thus hydrophilic and/or hydrophobic actives or flavours may be delivered by the particle of the invention. The hydrophobic actives or flavours can be carried in the core and the hydrophilic actives or flavours can be carried in the core when the core comprises a water-in-oil emulsion and/or in each of the layers.

The term “oil” is used to refer to fats that are liquid at normal room temperature, while the term “fat” is used to refer to fats that are solid at normal room temperature. Suitable oils or fats can be derived from vegetable, animal, mineral or synthetic sources. Thus the core can be in the form of a liquid or a solid. Any oil or fat may be used. Therefore the fat or oil may be selected from the group consisting of vegetable fat or oil, animal fat or oil, mineral fat or oil and synthetic fat or oil. Preferred vegetable oils are coconut oil, corn oil, cottonseed oil, canola oil (rapeseed oil), olive oil, palm oil, peanut oil (ground nut oil), safflower oil, sesame oil, soybean oil, sunflower oil, almond oil, cashew oil, hazelnut oil, macadamia oil, pecan oil, pistachio oil, walnut oil, acai oil, blackcurrant seed oil, borage seed oil, evening primrose oil, carob seed pods, amaranth oil, apricot oil, argan oil, avocado oil, babassu oil, ben oil, carob pod oil (algaroba oil), coriander seed oil, false flax oil (made of the seeds of camelina sativa), coriander seed oil, hemp oil, kapok seed oil, meadowfoam seed oil, mustard oil (pressed), okra seed oil, perilla seed oil, pine seed oil, poppyseed oil, prune kernel oil, pumpkin seed oil, quinoa oil, ramtil oil, rice bran oil, tea oil (camellia oil), thistle oil, wheat germ oil, castor oil, radish oil, ramtil oil, allanblackia oil and tung oil. Preferred animal oils are tallow oil and fish oil (for example cod liver oil).

The core may comprise an oil, a fat, a mixture of oil and fat, a water-in-oil emulsion or a mixture of oil and fat and water-containing or solid particles. Furthermore the core has a largest dimension of 10 nm to 200 μm, preferably 50 nm to 30 μm and can adopt any shape, such as a sphere, a rod, a disc, as well as other ill-defined shapes.

The release profile of actives and/or flavours from the interior of the coated particle of the invention can be optimised for a given purpose by controlling the size and nature of the core. For example the release profile may be optimised for sensorial reward or to improve efficacy, for example by enabling sustained release of an active, or to mitigate negative side-effects, such as skin irritancy, arising from a more intense release of active over a shorter time period.

The emulsifier may be any emulsifier. Preferably the emulsifier is a sucrose ester of formula (I):

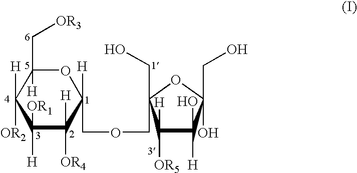

wherein R₁, R₂, R₃, R₄ and R₅ are independently from each other any non-toxic fatty acid, such as stearic, palmitic, oleic, lauric, erucic and other fatty acids. Specific examples of suitable sucrose esters are the mixtures of sucrose monostearate and sucrose distearate (especially the 70%:30%-mixture) or the mixture of sucrose monopalmitate and sucrose dipalmitate.

By polyanion is meant a charged molecule with a net charge of at least two negative charges. By polycation is meant a charged molecule with a net charge of at least two positive charges.

Suitable polyanions include naturally occurring as well as synthetic polyanions. Examples include alginate, carboxymethylamylose, carboxymethylcellulose, carboxymethyldextran, carrageenan, cellulose sulfate, chrondroitin sulfate, chitosan sulfate, dextran sulfate, gum arabic, gum tragacanth, guar gum, gellan gum, heparin, hyaluronic acid, pectin, amidated pectins, xanthan, proteins and glycoproteins such as mucins.

Suitable polycations include naturally occurring and synthetic polycations. Examples include chitosan, modified dextrans such as diethylaminoethyl-modified dextrans, hydroxymethylcellulose trimethylamine, lysozyme, polylysine, protamine sulfate, hydroxyethylcellulose trimethylamine and other proteins.

More generally suitable polyanions or polycations may also be cross-linkable. Also suitable polyanions or polycations may be linear or branched biopolymers. The term biopolymer is meant a polymer which adheres to a biological surface whether internal or external. Suitable biopolymers include:
Chitosan and its derivatives;
Derivatives of acrylic acid such as polyacrylic acid, carbomers (Carbopol® 980, 974 and 971 available from Noveon), polyacryl cyanoacrylates, ammonium methacrylates (Eudragit® RS, RL, NE30D available from Degussa) and polycarbophil (Noveon® Polycarbophil available from Noveon);
Cellulose derivatives such as sodium carboxymethyl cellulose, hydroxy ethyl cellulose and hydroxypropyl cellulose;
hyaluronic acid and its derivatives, such as sodium hyaluroniate;
Other polysaccarides such as carrageenan (kappa, iota), sodium alginate, gellan, polygalacturonic acid, pectin, xyloglucan gum, modified dextrans such as amino-conjugated dextran, and scleroglucan;
Thiomers such as polyacrylic acid cysteine conjugate, polycarbophil cysteine conjugate, chitosan cysteine conjugate and chitosan 4-thiobuthylamidine;
Polyvinyl pyrrolidone;
Poloxamers (Pluronics® range from BASF); and
Proteins such as lysozymes.

A further list of suitable biopolymers is provided in WO 2004/071489 A1 (page 4, lines 19-31).

The release profile of actives and/or flavours from the interior of the coated particle of the invention can also be optimised for a given purpose by changing the composition of the first innermost layer, the second middle layer coating and the third outermost layer. In particular the release properties of the layers can be tailored to respond to changes in temperature, salt levels, pH or the presence of particular enzymes present in, for example, the mouth. Thus the release profile may be optimised for sensorial reward or to improve efficacy, for example by enabling sustained release of an active, or to mitigate negative side-effects, such as skin irritancy, arising from a more intense release of active over a shorter time period.

The thickness of each layer may be 1 nm to 500 nm and thus the largest dimension of the coated particle may be 11 nm to 200 μm, preferably 50 nm to 30 μm. The thickness of each layer may be measured by using commonly known methods such as ellipsometry, dual polarisation interferometry, optical waveguide light spectroscopy, a quartz crystal microbalance, dynamic light scattering, atomic force microscopy (or other scratch test instruments using similar principles) or fluorescent confocal microscopy. The coated particle may adopt any shape, such as a sphere, a rod, a disc, as well as other ill-defined shapes.

The second middle layer may either have a net charge opposite to that of the first innermost layer or not. In the latter case, the second middle layer coats the first innermost layer through the action of extensive hydrophobic groups in the second middle layer.

The coated particle may comprise more than three layers, for example at least four layers, with successive layers consisting of either one or more polyanions or one or more polycations in alternating fashion, a fourth layer coating the third outermost layer, wherein the fourth layer coating consists only of one or more polyelectrolytes of opposing charge to that of the polyanions or polycations of the third outermost layer. Preferably the coated particle comprise 3 to 20 layers, more preferably 3 to 10 layers.

The release profile of actives and/or flavours from the interior of the coated particle of the invention can also be optimised for a given purpose by varying the number of layers with additional layers typically slowing release. Thus the release profile may be optimised for sensorial reward or to improve efficacy, for example by enabling sustained release of an active, or to mitigate negative side-effects, such as skin irritancy, arising from a more intense release of active over a shorter time period.

Usually a coated particle comprises 0.001 to 99.999, preferably 10 to 99.999, more preferably 20 to 99.999, most preferably 50 to 99.999% by weight core based on the total weight of the coated particle.

In another aspect of the invention, a product is provided selected from the group consisting of a food product, a home care product, a personal care product and a pharmaceutical product, wherein each product comprising a plurality of coated particles according to the invention.

By food product is meant any food product for animals or humans though preferably for humans. Thus suitable food products include any kind of drink or other liquid food product, snacks, candies and confections, cookies, fillings, toppings, dessert mixes, granola bars, energy bars, shelf stable powders, puddings, yogurts, frozen yogurts, ice creams, cereals, meal replacements, baked goods, pasta products, military rations, specially formulated foods for children, mayonnaise, salad dressings, sauces, dips, creams, gravies, spreads, soups, coffee whiteners and desserts.

Home care products include laundry detergents and fabric softeners particularly when incorporating perfumes.

Personal care products include skin creams, soaps, soap bars, bath and shower gels, shampoos, mousses, deodorants, antiperspirants, lipsticks, sunscreens and oral care products such as toothpastes and mouthwashes.

In another aspect of the invention, a process for the manufacture of the particle of the invention is provided comprising the steps of:
(a) applying the first innermost layer to the core thereby to form a core coated with the first innermost layer; then
(b) washing the core coated with the first innermost layer; then
(c) applying the second middle layer to the first innermost layer thereby to form a core coated with the first innermost layer and the second middle layer; then
(d) washing the core coated with the first innermost layer and the second middle layer; then
(e) applying the third outermost layer to the second middle layer thereby to form a particle; and then
(f) washing the particle; then optionally
(g) repeating the application and washing steps for successive layers.

Electrophoretic measurements, by determining the zeta-potential or/and the surface charge of the partly or wholly formed particles, are used to confirm the satisfactory application of each layer and that the charge of each layer is alternating. The application step is carried out by immersing the partly formed particle in an aqueous solution of the layer. The degree of application of each layer can be controlled by varying the pH and ionic strength of each solution. The washing step is carried out in a washing liquor of water or an aqueous solution of appropriate pH and ionic strength followed by separated of the partly or wholly formed particles from the washing liquor by, for instance, centrifugation. The degree of application of each layer can be controlled by varying the temperature of the washing liquor. The partly or wholly formed particles may be subject to more than one wash.

### BRIEF DESCRIPTION OF THE FIGURES

The invention will now be illustrated with reference to:

FIG. 1 shows a cryo-electron microscope image of a coated particle comprising palm oil core, and seven layers of successively sugar ester emulsifier and then alternating chitosan (2^{nd}, 4^{th}, 6^{th} layers) and purified porcine gastric mucin (3^{rd}, 5^{th}, 7^{th} layers) according to example 1;

FIG. 2 shows a cryo-electron microscope image of a palm oil droplet with only first innermost layer;

FIG. 3 shows sequential zeta potential measurements for each of the seven layers coating the palm oil core of example 1;

FIG. 4 shows a coated particle comprising corn oil core, and four layers of successively sugar ester emulsifier and then chitosan (2^{nd} and 4^{th} layers) and then pectin (3^{rd} layer) according to example 4;

FIG. 5 shows a cryo-electron microscope image of a corn oil droplet with only first innermost layer;

FIG. 6 shows sequential zeta potential measurements for each of the four layers coating the corn oil core of example 4;

FIG. 7 shows sequential zeta potential measurements for each of the five layers coating the toasted sesame oil core of example 6;

FIGS. 8a and 8b show the light microscopy images of the control and the particles in various stages of preparation as indicated with and without human whole saliva in accordance with example 6;

FIG. 9 shows images captured of the natural fluorescence of the coated particles at various stages of preparation as indicated on a human tongue after the samples were expectorated in accordance with example 6;

FIG. 10 shows the analysis of the intensity of fluorescence on the tongue immediately after the samples have been expectorated corresponding to the images in FIG. 9;

FIG. 11 shows the residence times for the coated particles at various stages of preparation illustrated in FIG. 9;

FIG. 12 shows zeta potential measurements for the various coated particles according to example 7;

FIG. 13 shows light microscopy images of the coated particles of FIG. 12 in the presence of human whole saliva;

FIG. 14 shows a schematic diagram of the APCI MS breath equipment; and

FIG. 15 shows the release of menthol from the coated particles of FIG. 12.

### DETAILED DESCRIPTION OF THE INVENTION

If not otherwise stated, any percentages are weight percentages and temperatures are given in Celsius.

### CryoSEM

CryoSEM was carried out by quick freezing samples using liquid nitrogen followed by evaporation of water. Quick freezing ensures minimal distortion of the structure of the sample. Further details of the technique can be found in “Interfacial structure of solid-stabilised emulsions studied by scanning electron microscopy”, Binks et al., Physical Chemistry Chemical Physics, 4 (15), 3727-3733, (2002).

### Zeta Potential

Electrophoretic mobility measurements were carried out on a Malvern ZetaSizer Nano series instrument with backscattering dynamic light scattering detection. The mobility data was used to calculate the zeta potential or surface charge density of the particle. Samples were prepared by dilution in deionised water to approximately 1% by weight oil in order to make measurements. 1 ml of the prepared sample was loaded into the cuvettes and the data obtained at 25° C. The results were analysed using the Malvern DTS (Dispersion Technology Software).

### Light Microscopy

Light microscopy of the coated particles was carried out using an Leitz Ortholux II light microscope using a bright field configuration. Images were obtained for the coated particles alone and after mixing the samples with human whole saliva.

### Oil Deposition and Clearance

In-vivo assessment of oil deposition was achieved from in-mouth imaging data collected using a fluorescent video endoscope that enables real-time images of the oil inside the oral cavity to be recorded. The images and data on deposition and clearance of coated particles in the mouth were obtained using the equipment and procedures outlined in “In-vivo visualisation of mouth-material interactions by video rate endoscopy”, Adams et al., Food Hydrocolloids, 21, 986-995, (2007). An excitation wavelength of 488 nm was used to excite the natural fluorescence of sesame seed oil comprising the core of coated particles assessed from a 15% suspension. The subjects were two healthy male volunteers Who were asked to clean their mouth and tongue one hour before the start of the trial in order to remove any residual food bits that might give rise to high background scatter. After a background scan of the tongue was taken, the volunteers were then asked to process a 4 ml sample containing an aqueous dispersion of the coated particles by swilling round the mouth for 32 seconds. The sample was then expectorated and digital video images of the product residue in-mouth were collected by scanning the surface of the tongue over a prescribed path (right, back and left) over 50 seconds to gain a picture of the product distribution in the oral cavity. The scanning was repeated at 2.5 minute intervals for 25 minutes to enable an assessment of the clearance rate of the residue from the mouth. The video thereby collected was then analysed frame by frame. The average mean intensity (I) over the appropriate frames was then calculated within each region (right, back and left) to give a measure of the total residue amount on the tongue and the standard deviation in the frame to frame intensity was used as an indication of scatter in data. This was then plotted as a function of time (t) to determine the residence time for the residue T, and initial deposited amount I₀ where I=I₀exp(−t/τ).

### Flavour Release

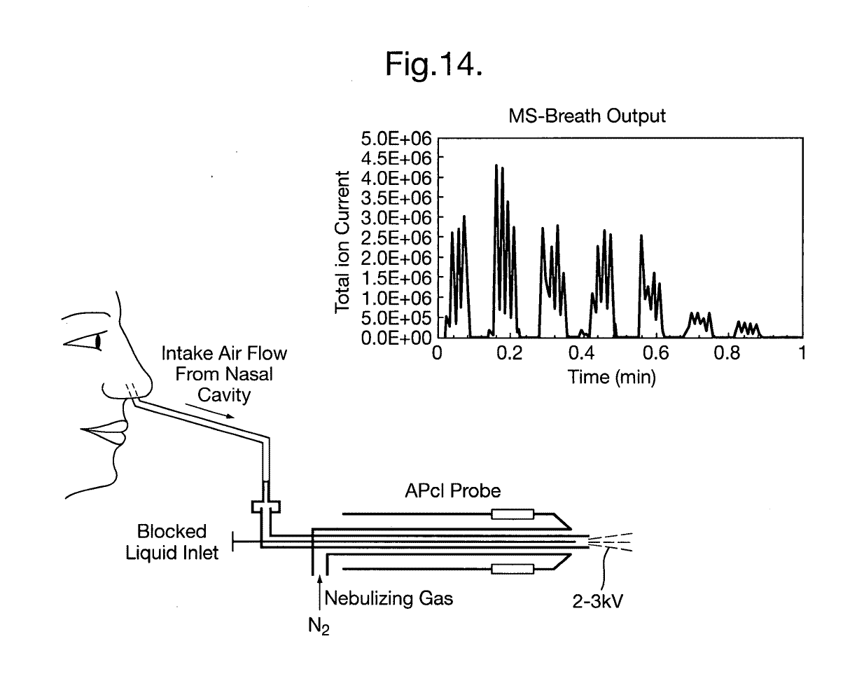

Flavour release was measured by an atmospheric pressure chemical ionisation mass spectrometry (APCI-MS) breath technique using a Navigator mass spectrometer fitted with APcl interface and Xcalibur software. This technique measures the release of volatile (flavour) molecules into the nasal cavity from a food sample during a chewing process The technique involves gently sucking exhaled air from the nose into the mass spectrometer where the volatiles are detected as protonated [M+H]⁺ ions. The subject sipped 2.5 ml of the sample and swilled it around in the mouth, swallowed it and then chewed at a rate of approximately 1 chew per second with a respiratory rate of between 6-8 breaths per minute for 5 minutes. After this time the panelist washed their mouth with water before taking a second sample. Each product was sampled in duplicate or triplicate. The obtained chrmoatograms were integrated and the mean area counts of each exhalation peak were plotted as a function of time and the results presented as time-intensity profiles. Results were obtained in duplicate. Further details of the technique can be found in “Atmospheric pressure chemical ionisation mass spectrometry for in-vivo analysis of volatile flavour release”, Blake et al., Food Chem., 71(3), 327-338, (2000) and a schematic diagram of the apparatus is shown in FIG. 14.

### EXAMPLE 1

Coated particle comprising palm oil core, and seven layers of successively sugar ester emulsifier and then alternating chitosan (2^{nd}, 4^{th} and 6^{th} layers) and purified porcine gastric mucin (3^{rd}, 5^{th} and 7^{th} layers) layers.

### Fabrication of the Coated Particle

### Solution Preparation

The chitosan solution was prepared by dispersing 0.1% by weight of powdered chitosan (ChitoClear®, Primex Ingredients ASA, Norway) in 100 mM NaCl (99.98%, Riedel-de-Haen, UK) solution in deionised water (Millipore system—deionized water resistivity of 18.2 MOhm and filtered through 0.2 μm filter). The pH was adjusted to 3.0 using H₃PO₄ (80%, Riedel-de-Haen, UK). The system was mixed for 30 minutes until full dissolution of the chitosan was achieved.

The mucin solution was obtained by dispersing 0.1% by weight of mucin (porcine gastric mucin from “Saliva Orthana®”, a saliva substitute, A/S Orthana Kemisk Fabrik, Kastrup, Denmark) into 100 mM NaCl solution in deionised water. The solution was mixed for 30 minutes using a magnetic stirrer to dissolve fully all the mucin. Both chitosan and mucin solutions were cooled down to 0° C. and stored in an ice bath before use.

1% by weight of Sugar Ester P-1670 (RYOTO Sugar Ester, Mitsubishi-Kagaku Foods Corporation, Japan) was dispersed in deionised water, heated slightly (to about 70° C.) and mixed gently with a magnetic stirrer for at least 3 hours.

### Primary Emulsion Preparation (First Innermost Layer)

15% by weight of melted palm oil (at about 50° C.) was poured into the hot (at about 70° C.) sugar ester solution over the course of 1 minute and the emulsion mixed gently by magnetic stirrer for 40 minutes whilst maintaining the temperature at 70° C. After that the coarse hot emulsion was mixed with a Silverson L4R mixer for 1 minute to obtain a fine emulsion.

### Layer-by-Layer (LBL) Deposition (Layer Two and Subsequent Layers)

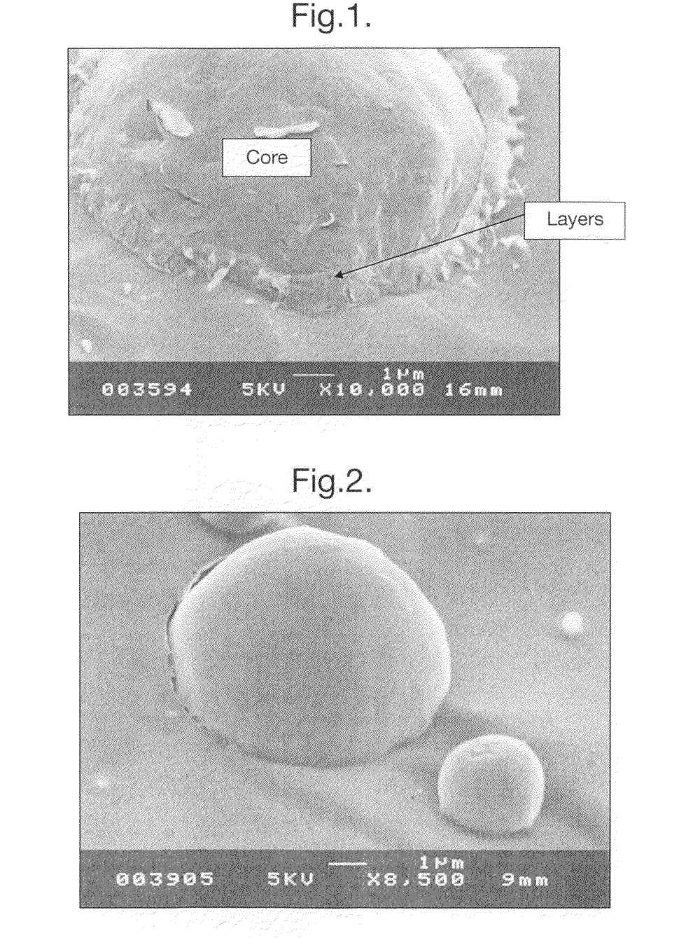

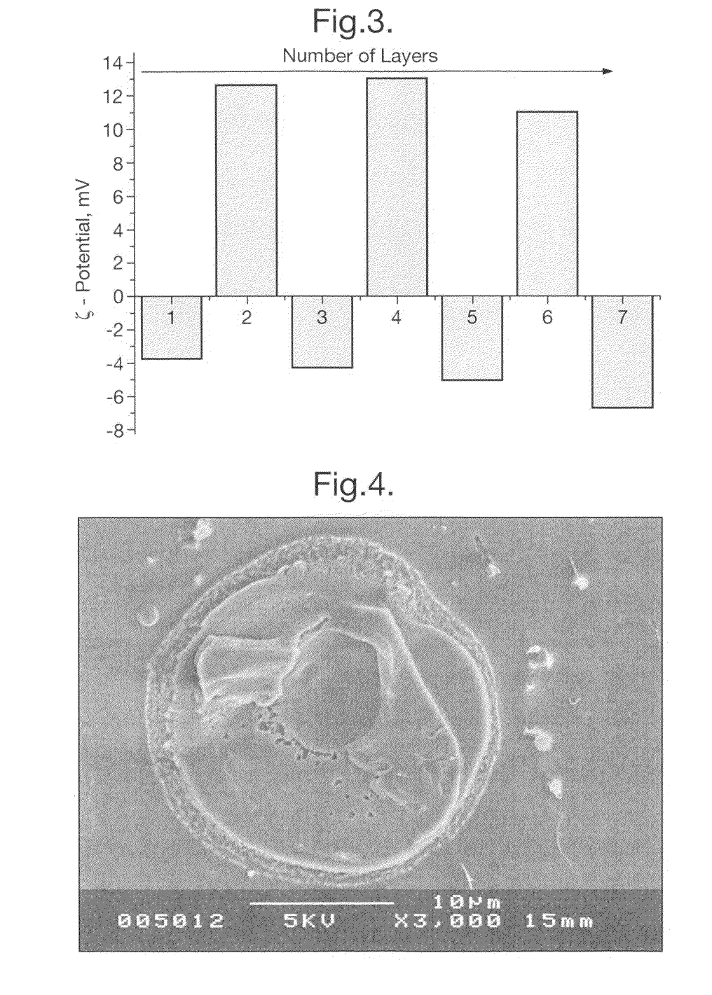

After the primary emulsion was prepared, it was poured into an equal volume of ice-cold (at about 0° C.) deionised water. It was cooled for 5 to 7 minutes and then centrifuged at 600 g in a Sorval 5CRC centrifuge for 5 minutes. This speed and the centrifugation time were kept constant for all centrifugation operations mentioned below. The temperature during centrifugation and during all the layer-by-layer deposition was 0° C. After centrifugation the supernatant was discarded and chitosan solution added. The emulsion was re-dispersed and held at 0° C. for 30 to 40 minutes in order to adsorb the chitosan. The emulsions prepared by this method were always easily re-dispersible. After deposition of chitosan, the emulsion was centrifuged, the supernatant discarded and a new portion of 0° C. cooled deionised water added. Then the emulsion was re-dispersed, centrifuged again and the supernatant was discarded (the “washing procedure”). After the “washing procedure” mucin solution was added. The emulsion was re-dispersed and left for 30 to 40 minutes to adsorb the mucin. After this the “washing procedure” was repeated. Additional chitosan and mucin layers were subsequently deposited in a sequential manner using the same method. This procedure was repeated until six layers had been deposited by layer-by-layer deposition. The emulsion was then washed twice with deionised water and stored at 0° C. FIG. 1 shows a cryo-electron microscope image of the resulting coated particle. For the purposes of comparison, FIG. 2 serves shows a cryo-electron microscope image of a palm oil droplet with only first innermost layer. FIG. 3 shows sequential zeta potential measurements for each of the seven layers coating the palm oil core.

### EXAMPLE 2

Coated particle comprising castor oil core, and five layers of successively sugar ester emulsifier and then alternating chitosan (2^{nd} and 4^{th} layers) and purified porcine gastric mucin (3^{rd} and 5^{th} layers) layers.

### Fabrication of the Coated Particle

### Solution Preparation

The chitosan and sugar ester solutions were prepared in the same way as used in example 1. The mucin solution was obtained by dispersing 0.1% by weight of mucin (porcine gastric mucin from “Saliva Orthana®”, a saliva substitute, A/S Orthana Kemisk Fabrik, Kastrup, Denmark) in 1 mM NaCl solution in deionised water. The pH was adjusted to 5.0 using H₃PO₄ (80%, Riedel-de-Haen, UK) and the solution mixed for 30 minutes using a magnetic stirrer to dissolve fully all the mucin.

### Primary Emulsion Preparation (First Innermost Layer)

20% by weight of castor oil was poured into the hot (about 70° C.) sugar ester solution over the course of 1 minute. The emulsion was mixed gently using a magnetic stirrer for 40 minutes. The temperature was maintained at 70° C. during this period. The coarse, hot emulsion was then mixed with a Silverson L4R mixer for 1 minute to obtain a fine emulsion.

### Layer-by-Layer (LBL) Deposition (Layer Two and Subsequent Layers)

The method is that as described for example 1 except that the temperature of the deionised water, chitosan solution and mucin solution was that of room temperature. Also centrifugation was carried out at 1800 g and at room temperature and storage of the final product was at chill temperature (4° C.).

### EXAMPLE 3

Coated particle comprising corn oil core, and three layers of successively sugar ester emulsifier and then chitosan (2^{nd} layer) and then pectin (3^{rd} layer).

### Fabrication of the Coated Particle

### Solution Preparation

The chitosan solution was prepared as described in example 1 except pH adjustment was carried out using acetic acid. The solution was stored at chill temperature (4° C.).

The pectin (Genu® pectin AS confectionery, CP Kelco) solution was prepared by dispersing 1% by weight of powdered pectin into deionised water. The mixture was stirred for 2 hours using a magnetic stirrer to fully dissolve all the pectin. The solution was stored at chill temperature (4° C.).

The sugar ester solution was prepared as described in example 1.

### Primary Emulsion Preparation (First Innermost Layer)

The preparation was the same as described in example 2 except 10% by weight of corn oil was used instead of 20% by weight castor oil.

### Layer-by Layer (LBL) Deposition (Layer Two and Subsequent Layers)

The process was analogous to that described for example 1 except that the centrifuge time was 3 minutes and not 5 minutes. Also the final product was stored at chill temperature (4° C.) rather than 0° C.

### EXAMPLE 4

Coated particle comprising corn oil core, and four layers of successively sugar ester emulsifier and then alternating chitosan (2^{nd} and 4^{th} layers) and then pectin (3^{rd} layer) layers.

### Fabrication of the Coated Particle

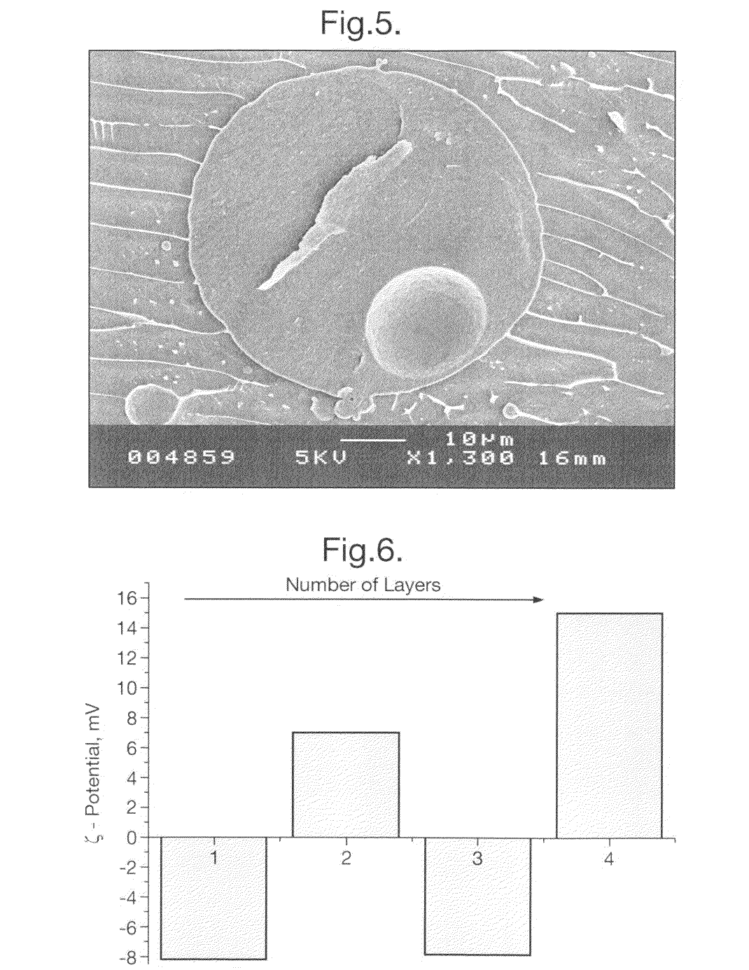

This example was produced using an analogous process to that described in example 3 but with an additional layer of deposited chitosan. FIG. 4 shows a cryo-electron microscope image of the resulting coated particle. For the purposes of comparison, FIG. 5 serves shows a cryo-electron microscope image of a corn oil droplet with only first innermost layer. FIG. 6 shows sequential zeta potential measurements for each of the four layers coating the palm oil core.

### EXAMPLE 5

Coated particle comprising corn oil core, and four layers of successively sugar ester emulsifier and then alternating chitosan (2^{nd} and 4^{th} layers) and then purified porcine gastric mucin (3^{rd} layer) layers.

### Fabrication of the Coated Particle

### Solution Preparation

The chitosan solution and sugar ester solution were prepared as described in examples 3 and 1 respectively.

The mucin solution was obtained by dispersing 0.1% by weight of mucin (porcine gastric mucin from “Saliva Orthana®”, a saliva substitute, A/S Orthana Kemisk Fabrik, Kastrup, Denmark) into deionised water with mixing for 30 minutes, using a magnetic stirrer, to fully dissolve all the mucin. The solution was stored at chill temperature (4° C.) until used.

### Primary Emulsion Preparation (First Innermost Layer)

The primary emulsion was prepared by a method analogous to that described in example 3.

### Layer-by-Layer (LBL) Deposition (Layer Two and Subsequent Layers)

Layer-by-layer deposition was achieved using an analogous process to that described in example 4.

### EXAMPLE 6

Coated particle comprising toasted sesame seed oil core, and up to five layers of successively sugar ester emulsifier and then alternating chitosan (2^{nd} and 4^{th} layers) and then pectin (3^{rd} and 5^{th} layers) layers.

### Fabrication of the Coated Particle

### Solution Preparation

The chitosan solution was prepared by dispersing 0.2% by weight. of powdered chitosan (ChitoClear®, Primex Ingredients ASA, Norway) in deionised water (Millipore system—deionized water resistivity of 18.2 MOhm and filtered through 0.2 μm filter) with 0.1% by weight potassium sorbate. The pH was adjusted to 3.0 using acetic acid. The ingredients were mixed for 30 minutes in order to achieve full dissolution. The solution was stored at chill temperature (4° C.) until used.

The pectin (Genu® pectin AS confectionery, CP Kelco) solution was prepared by dispersing 1% by weight of powdered pectin into deionised water with 0.1% by weight potassium sorbate It was mixed for 2 hours using a magnetic stirrer to dissolve all the pectin. The solution was stored at chill temperature (4° C.) until used.

The sugar ester solution was prepared as described in example 1.

### Primary Emulsion Preparation (First Innermost Layer)

30% by weight of toasted sesame seed oil was poured into a solution of the sugar ester (Ryoto R-1670, Mitsubishi-Kagaku Foods Corporation, Japan), 0.05% Tween 60 (Uniquema polysorbate 60) held at above 70° C. The emulsion was mixed gently with a magnetic stirrer for 1 minute, whilst maintaining the temperature above 70° C. The resulting coarse emulsion was mixed with a Silverson L4R mixer for 1 minute at high speed to obtain a fine emulsion. The fine emulsion was then held for 2 minutes at 85° C. before being cooled rapidly in iced water. The resulting suspension was then stored at chill temperature (4° C.).

### Layer-by-Layer (LBL) Deposition (Layer Two and Subsequent Layers)

Layer-by-layer deposition was achieved using an analogous process to that described in example 3.

### Preparation of the Final Emulsion

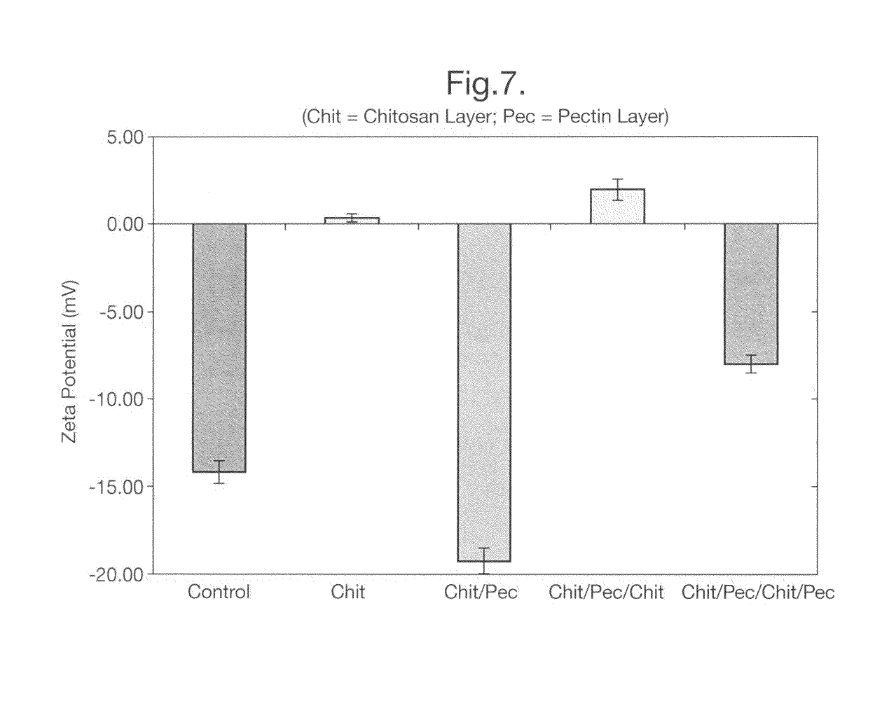

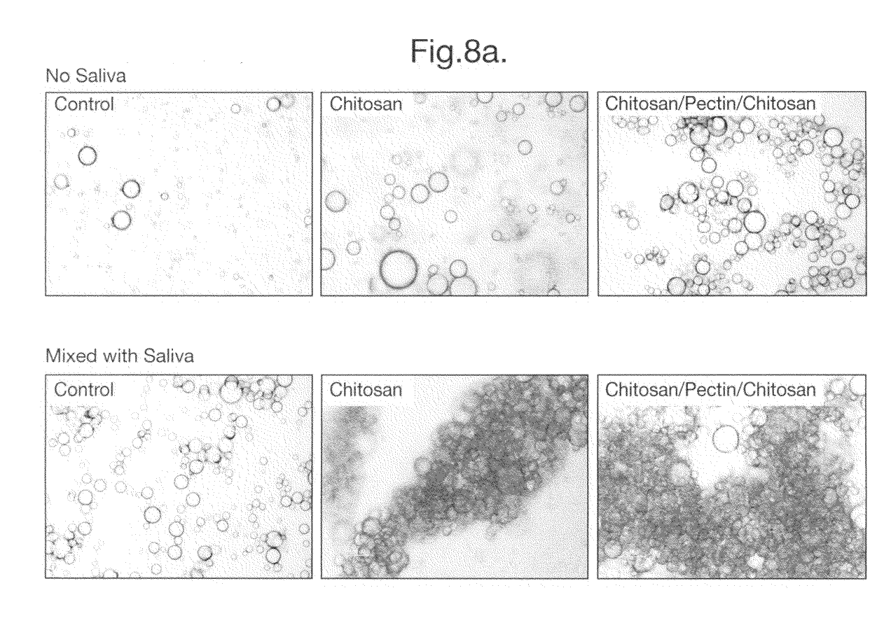

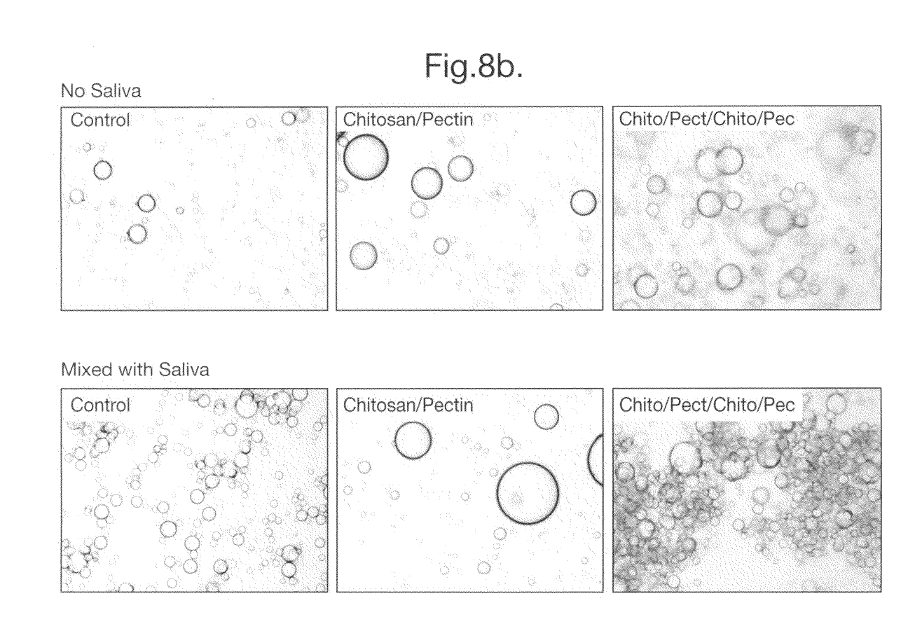

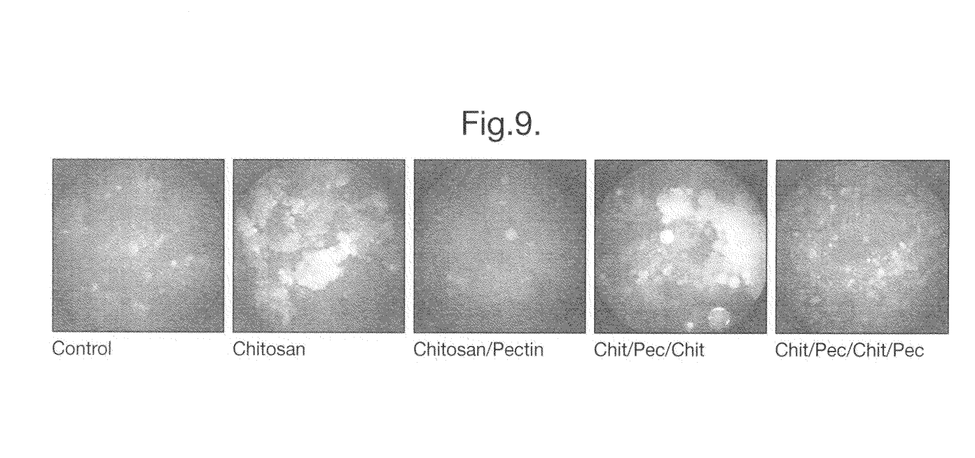

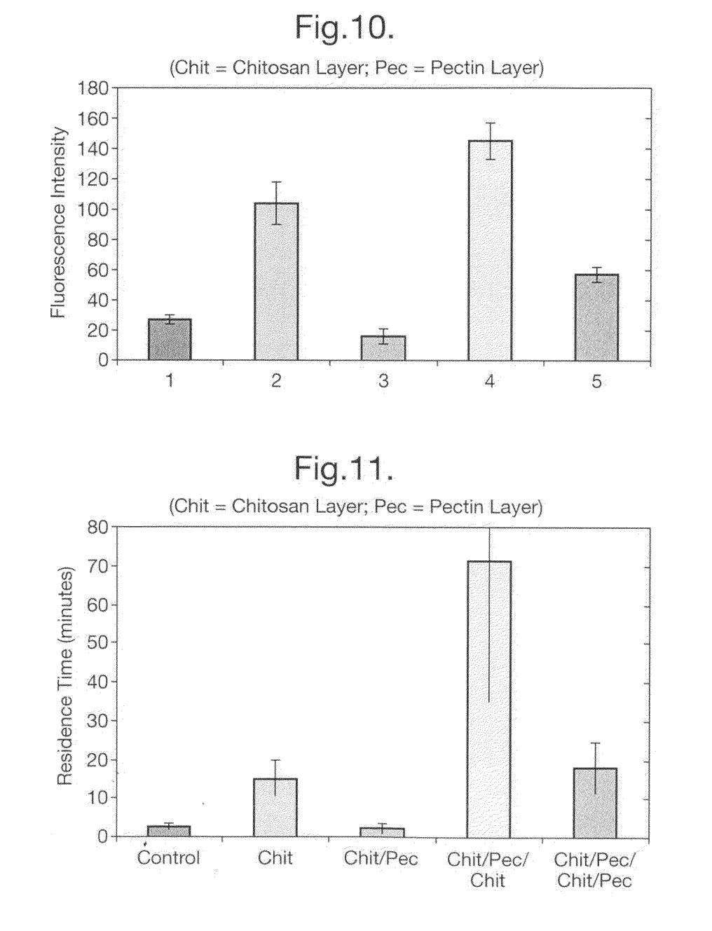

The final product was prepared by mixing equal volumes of the coated particle in the form of an emulsion (30% oil content) with an equal volume of a 45% by weight maltodextrin (Roquette 2DE maltodextrin), 0.2% by weight citric acid and 0.1% by weight potassium sorbate solution. The emulsion was carefully dispersed and stored at chill temperature (4° C.) until further use. The final composition of the emulsion was 15% by weight toasted sesame seed oil, 22.5% by weight maltodextrin, 0.1% by weight citric acid and 0.1% by weight potassium sorbate. The pH of the emulsion was 3.0. Maltodextrin was used as a thickener to prevent creaming of the emulsion. Citric acid and potassium sorbate were used as preservatives. FIG. 7 shows sequential zeta potential measurements for each of the five layers coating the toasted sesame oil core. The chitosan layers are more positively charged that the pectin layers. FIGS. 8a and 8b show the light microscopy images of the control and the particles in various stages of preparation as indicated with and without human whole saliva. It was observed that when the terminal layer is chitosan, the interaction with saliva produced visible flocculation and when conversely when the terminal layer is pectin the interaction with saliva is very much reduced. FIG. 9 shows images captured of the natural fluorescence of the coated particles at various stages of preparation as indicated on a human tongue after the samples were expectorated. Inspection of these images suggests that when the terminal layer is chitosan, more oil remains behind on the tongue. FIG. 10 shows the analysis of the intensity of fluorescence on the tongue immediately after the samples have been expectorated corresponding to the images in FIG. 9. These results show the improved deposition achieved by the particles whose terminal layer is chitosan and in particular by the chitosan/pectin/chitosan coated particles. FIG. 11 shows the residence times for the coated particles at various stages of preparation illustrated in FIG. 9. The results mirror those of FIG. 10.

### EXAMPLE 7

Coated particle comprising palm oil core, and up to five layers of successively sugar ester emulsifier and then various polyelectrolytes.

### Fabrication of the Coated Particle

### Solution Preparation

The chitosan solution was prepared by dispersing 0.2% by weight of powdered chitosan (ChitoClear®, Primex Ingredients ASA, Norway) in deionised water (Millipore system—deionized water resistivity of 18.2 MOhm and filtered through 0.2 μm filter) with 0.1% by weight potassium sorbate and 150 mM NaCl. The pH was adjusted to 4.0 using 2 ml of phosphoric acid. The ingredients were mixed for 30 minutes in order to achieve full dissolution. The solution was stored at chill temperature (4° C.) until used.

The pectin solution was prepared and stored in the same way as set forth in example 6.

The lysozyme (Belovo SA) solution was prepared by dissolving 1% by weight lysozyme in deionised water with 0.1% by weight potassium sorbate. The solution was stirred to dissolve the lysozyme. The solution was stored at chill temperature (4° C.) until used.

The mucin solution was obtained by dispersing 0.1% by weight of mucin (porcine gastric mucin from “Saliva Orthana®”, a saliva substitute, A/S Orthana Kemisk Fabrik, Kastrup, Denmark) in deionised water containing 0.1% by weight potassium sorbate. The solution was mixed for 30 minutes using a magnetic stirrer to fully dissolve the mucin. The solution was stored at chill temperature (4° C.) until used.

The sugar ester solution was prepared as described in example 1.

### Primary Emulsion Preparation (First Innermost Layer)

The method used to prepare the primary emulsion was analogous to that used in example 6 except that the palm oil content was 20% by weight. The palm oil used was Loders Croklaan Ceamelt 900. For flavoured samples, menthol (Sigma FCC Kosher DL-menthol code: W266507) was also added to the molten oil prior to making the emulsion. The level used was 0.08 g of menthol per 100 g of palm oil.

### Layer-by-Layer (LBL) Deposition (Layer Two and Subsequent Layers)

The layer-by-layer preparation method employed was analogous to that described in example 6. For menthol flavoured coated particles, the wash water was however saturated with menthol to reduce partitioning of the flavour into the water phase.

### Preparation of the Final Emulsion

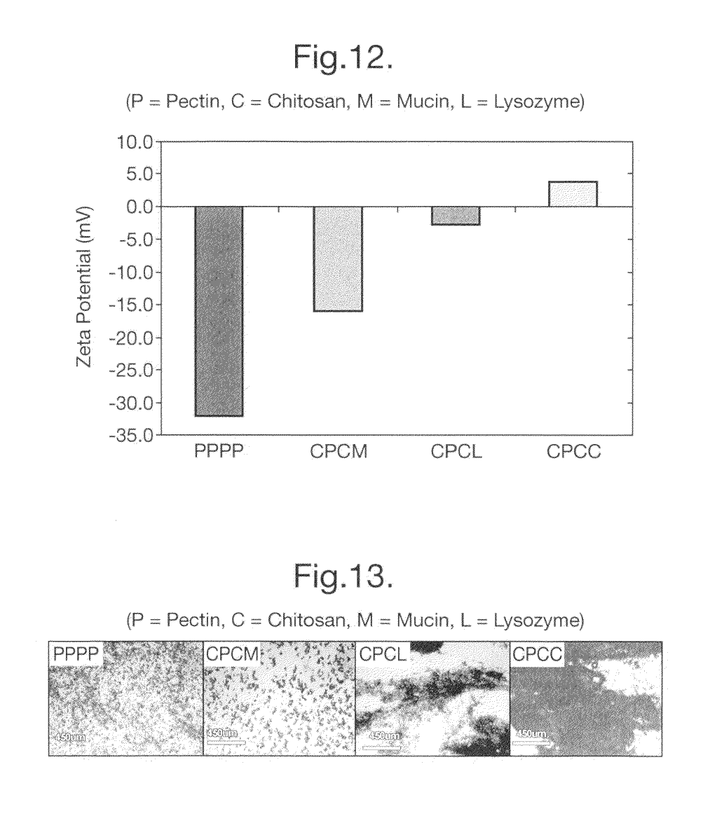

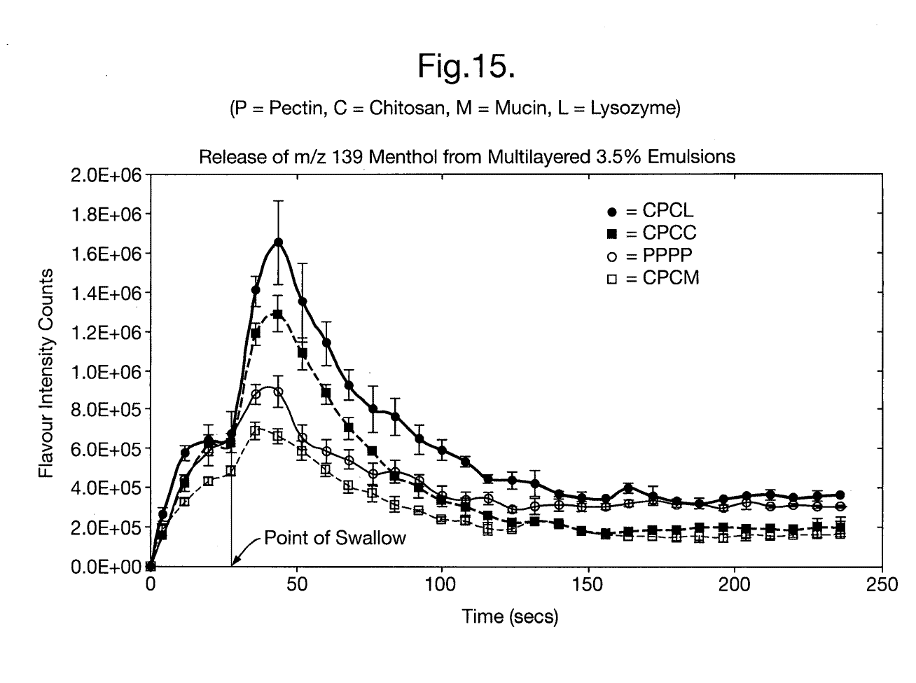

The final product was prepared by mixing equal volumes of the coated particle in the form of an emulsion (20% by weight oil content) with a 1% by weight guar gum (THI), 0.1% by weight potassium sorbate and an 85% by weight phosphoric acid solution (added at a level of 2 ml per litre). The solution pH was 4.0. The product was carefully mixed and stored at chill temperature (4° C.) until further use. The final composition of the product was 10% by weight palm oil, 0.5% by weight guar gum and 0.1% by weight potassium sorbate. The pH of the product was 4.0. Guar was used as a thickener to prevent creaming of the emulsion and potassium sorbate was used as a preservative. For the APCI-MS breath studies the product was further diluted with a 0.5% by weight guar gum and 0.1% by weight potassium sorbate solution with phosphoric acid added as before. The final pH of the product was 4.0 and the final palm oil concentration 3.5% by weight. FIG. 12 shows zeta potential measurements for the various coated particles. It can be observed that when the terminal layer is a positively charged polymer at pH 4, the zeta potential of the coated particle is more positive. FIG. 13 shows light microscopy images of the coated particles of FIG. 12 in the presence of human whole saliva. It appears that more interaction occurs with human whole saliva with lysozyme and chitosan terminated coated particles. FIG. 15 shows the release of menthol from the coated particles of FIG. 12. The results show that increasing the interaction between the coated particles and human whole saliva leads to increased flavour release as measured by the by the APCI-MS-Breath technique.

1. A coated particle comprising a core, a first innermost layer coating the core, a second middle layer coating the first innermost layer and a third outermost layer coating the second middle layer, wherein the core comprises at least a water-in-oil emulsion or a fat and/or oil, wherein the first innermost layer comprises at least one emulsifier and, wherein the second middle layer comprises either one or more polyanions or one or more polycations and the third outermost layer comprises one or more polyelectrolytes only of opposing charge to that of the polyanions or polycations of the second middle layer.

2. A coated particle according to claim 1, wherein the core is a liquid or a solid.

3. A coated particle according to claim 1, wherein the core comprises an oil, a fat, a mixture of oil and fat, a water-in-oil emulsion or a mixture of oil and fat and water-containing or solid particles.

4. A coated particle according to claim 1, wherein the core has a largest dimension of 10 nm to 200 μm, preferably 50 nm to 30 μm.

5. A coated particle according to claim 1, wherein the fat or oil is selected from the group consisting of vegetable fat or oil, animal fat or oil, mineral fat or oil and synthetic fat or oil.

6. A coated particle according to claim 5, wherein the fat or oil is selected from the group consisting of coconut oil, corn oil, cottonseed oil, canola oil, olive oil, palm oil, peanut oil, safflower oil, sesame oil, soybean oil, sunflower oil, almond oil, cashew oil, hazelnut oil, macadamia oil, pecan oil, pistachio oil, walnut oil, acai oil, blackcurrant seed oil, borage seed oil, evening primrose oil, carob seed pods, amaranth oil, apricot oil, argan oil, avocado oil, babassu oil, ben oil, carob pod oil, coriander seed oil, false flax oil, coriander seeds, hemp oil, kapok seed oil, meadowfoam seed oil, mustard oil, okra seed oil, perilla seed oil, pine seed oil, poppyseed oil, prune kernel oil, pumpkin seed oil, quinoa oil, ramtil oil, rice bran oil, tea oil, thistle oil, wheat germ oil, castor oil, radish oil, ramtil oil, allanblackia oil, tung oil, tallow oil and fish oil.

7. A coated particle according to claim 1, wherein the emulsifier is a sucrose ester of formula (I) 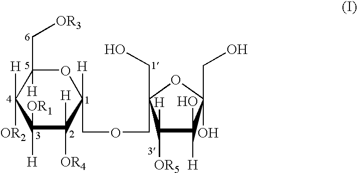
wherein R₁, R₂, R₃, R₄ and R₅ are independently from each other any non-toxic fatty acid, such as stearic, palmitic, oleic, lauric, erucic and other fatty acids.

8. A coated particle according to claim 1, wherein the polyanion is selected from the group consisting of alginate, carboxymethylamylose, carboxymethylcellulose, carboxymethyldextran, carageenan, cellulose sulfate, chrondroitin sulfate, chitosan sulfate, dextran sulfate, gum arabic, guar gum, gellan gum, heparin, hyaluronic acid, pectin, amidated pectines, xanthan, proteins and glycoproteins.

9. A coated particle according to claim 1, wherein the polycation is selected from the group consisting of chitosan, modified dextrans, hydroxymethylcellulose trimethylamine, lysozyme, polylysine, protamine sulfate, hydroxyethylcellulose trimethylamine and proteins.

10. A coated particle according to claim 1 comprising 0.001 to 99.999% by weight core based on the total weight of the coated particle.

11. A coated particle according to claim 1, the largest dimension of the coated particle is 11 nm to 200 μm.

12. A process for the manufacture of a particle according to claim 1 comprising the steps of
(a) applying the first innermost layer to the core thereby to form a core coated with the first innermost layer; then
(b) washing the core coated with the first innermost layer; then
(c) applying the second middle layer to the first innermost layer thereby to form a core coated with the first innermost layer and the second middle layer; then
(d) washing the core coated with the first innermost layer and the second middle layer; then
(e) applying the third outermost layer to the second middle layer thereby to form a particle; and then
(f) washing the particle; then optionally
(g) repeating the application and washing steps for successive layers.

13. A product selected from the group consisting of a food product, a home care product, a personal care product and a pharmaceutical product, wherein each product comprising a plurality of coated particles according to claim 1.
